(19) United States
(12) Patent Application Publication
(10) Pub. No.: US 2024/0317674 A1
(43) Pub. Date: Sep. 26, 2024

(54) HYDROGENATION OF AROMATIC AMINES FROM PU DECOMPOSITION PROCESSES

(71) Applicant: Evonik Operations GmbH, Essen (DE)

(72) Inventors: Stephan KOHLSTRUK, Gladbeck (DE); Thomas Rosen, Duelmen (DE); Roland Hubel, Essen (DE); Ralph Marquardt, Frankfurt/Main (DE); Torsten Panitzsch, Henstedt-Ulzburg (DE)

(73) Assignee: Evonik Operations GmbH, Essen (DE)

(21) Appl. No.: 18/575,878

(22) PCT Filed: Jun. 28, 2022
(86) PCT No.: PCT/EP2022/067707

§ 371 Date: Jan. 1, 2024

(30) Foreign Application Priority Data
Jul. 2, 2021 (EP) 21183456.9

(51) Int. Cl. C07C 209/72; C07C 209/62; C07C 209/86

(52) U.S. Cl. CPC C07C 209/72; C07C 209/62; C07C 209/86

(57) ABSTRACT

A process for catalytic hydrogenation of aromatic amines can be performed. The aromatic amines can be methylenedianiline and/or tolylenediamine. The ring-hydrogenated equivalents can be obtained by contacting an input product, which have the aromatic amines and impurities, such as, alcohols, glycols, polyols, organic acids and/or water, with hydrogen in the presence of a catalyst. The catalyst can be platinum, palladium, rhodium, ruthenium, nickel, cobalt and/or iron. The catalyst is applied to a support, wherein the input product that has aromatic amines result from a PU decomposition process and contains impurities from this decomposition process.

The invention is in the field of amines and polyurethane recycling. It especially describes a process for the catalytic hydrogenation of aromatic amines, such as methylenedianiline and tolylenediamine or mixtures thereof, wherein these amines result from polyurethane decomposition processes and contain residual impurities from this process.

On account of their exceptional mechanical and physical properties, polyurethanes find use in a very wide variety of sectors. A particularly important market for a very wide variety of types of polyurethanes is the polyurethane foams sector. Polyurethanes (PU) are for the purposes of the present invention all reaction products derived from isocyanates, in particular from polyisocyanates, and appropriately isocyanate-reactive molecules, in particular polyols. They also include inter alia polyisocyanurates, polyureas and isocyanate or polyisocyanate reaction products containing allophanates, biurets, uretdiones, uretonimines or carbodiimides.

Polyurethanes are now so widespread worldwide that recycling is becoming increasingly important for these materials too. Various decomposition processes for recovery of polyurethane wastes therefore already exist in the prior art. The known chemical processes such as hydrolysis, for example described in U.S. Pat. No. 5,208,379, glycolysis, acidolysis, aminolysis, hydrogenolysis, solvolysis and similar processes seek to effect depolymerization at a molecular level.

Also generated in the context of such polyurethane decomposition processes are aromatic amines which typically also comprise impurities originating from the decomposition process, for example alcohols, glycols, polyols, organic acids, tertiary amines, quaternary amines, aldehydes and/or water.

There is a need to effect optimal recovery of such starting material containing aromatic amines, such as methylenedianiline and/or tolylenediamine, which results from polyurethane decomposition processes and is contaminated with the byproducts from the decomposition process. Particular efforts have been made to obtain aliphatic amines from such contaminated but cost-effective starting materials.

This object is achieved by the subject matter of the invention This is a process for catalytic hydrogenation of aromatic amines, preferably comprising methylenedianiline and/or tolylenediamine, to afford their ring-hydrogenated equivalents comprising contacting an input product, which comprises the aromatic amines and up to 25% by weight, preferably up to 15% by weight, more preferably up to 10% by weight, particularly preferably up to 5% by weight, of impurities, such as in particular, alcohols, glycols, polyols, organic acids, tertiary amines, quaternary amines, aldehydes and/or water, the % by weight values being based on the input product comprising the aromatic amines, preferably methylenedianiline and/or tolylenediamine, with hydrogen in the presence of a catalyst, preferably comprising platinum, palladium, rhodium, ruthenium, nickel, cobalt and/or iron, in particular rhodium and/or ruthenium, applied to a fixed bed support, wherein the input product comprising aromatic amines results from a polyurethane decomposition process and contains impurities from this decomposition process.

The input product comprising the aromatic amines, preferably methylenedianiline and/or tolylenediamine, thus contains ≥75% by weight, preferably ≥85% by weight, more preferably ≥90% by weight, particularly preferably ≥95% by weight, in particular ≥98% by weight, of aromatic amines, preferably comprising methylenedianiline and/or tolylenediamine, and ≤25% by weight, preferably ≤ 15% by weight, more preferably ≤10% by weight, particularly preferably ≤5% by weight, in particular ≤2% by weight, of impurities, such as in particular alcohols, glycols, polyols, organic acids, tertiary amines, quaternary amines, aldehydes and/or water. A lower limit for these impurities may preferably be values such as for example 0.01% by weight, for example 0.1% by weight, for example 1% by weight, for example 3% by weight, for example 5% by weight, for example 10% by weight, for example 15% by weight or for example 20% by weight. An upper limit for the aromatic amines, preferably methylenedianiline and/or tolylenediamine, may preferably be values such as for example 99.99% by weight, for example 99.9% by weight, for example 99% by weight, for example 97% by weight, for example 95% by weight, for example 90% by weight, for example 85% by weight or for example 80% by weight. The % by weight values are in each case based on the input product comprising the aromatic amines, preferably methylenedianiline and/or tolylenediamine.

This subject matter is associated with several advantages. It makes it possible to hydrogenate a cost-effective and circular methylenedianiline or tolylenediamine-containing starting material which is additionally contaminated with byproducts from PU decomposition processes. The catalyst system may be reused over a relatively lengthy period. The cycloaliphatic amines obtainable in the catalytic hydrogenation of aromatic amines, such as unsubstituted or substituted cyclohexylamines and dicyclohexylamines, may be used for the production of polyamide and polyurethane resins, as hardeners for epoxy resins and as raw materials for producing plastic and rubber additives and corrosion inhibitors.

EP1604972A1 already discloses a process for hydrogenation of methylenedianiline. In the context of the present invention it has surprisingly been found that such a technology may also be used in particularly advantageous fashion on the contaminated aromatic amines from PU decomposition processes.

The process according to the invention employs a catalyst, preferably comprising platinum, palladium, rhodium, ruthenium, nickel, cobalt and/or iron, preferably comprising rhodium and/or ruthenium, in particular ruthenium, applied to a support.

The use of rhodium and/or ruthenium is particularly preferred. This corresponds to a particularly preferred embodiment of the invention. In a further very particularly preferred embodiment ruthenium is employed without rhodium.

In a further very particularly preferred embodiment of the invention ruthenium supported on aluminum oxide (Al₂O₃) is employed as catalyst. Aluminum oxide is a classical support material and is also commercially available as such a support material. Corresponding catalysts are commercially available for example as Noblyst® from Evonik. Ruthenium supported on aluminum oxide in this way results in very particularly advantageous results in the context of the present invention.

A further preferred embodiment of the invention is in effect when in the context of the abovementioned preferred embodiment the weight ratio of ruthenium to aluminum oxide is 4 to 25 parts by weight of ruthenium per 100 parts by weight of aluminum oxide. The use of ruthenium without rhodium, as described above, is very particularly preferred.

However, in a departure therefrom it is also possible to employ rhodium and ruthenium together in the context of another preferred embodiment.

When rhodium and ruthenium are employed, which is preferred, the weight ratio of rhodium to ruthenium is in particular 0.01 to 20 parts of rhodium per part of ruthenium.

In a preferred embodiment of the invention the catalyst/the catalyst system thus comprises rhodium and ruthenium and the catalyst system is preferably composed of a physical mixture of these two components. In particular, the catalyst system is a physical mixture of rhodium on a fixed bed support, preferably lithium aluminate, and ruthenium on a fixed bed support, preferably lithium aluminate. This corresponds to a preferred embodiment of the invention.

When rhodium is present in the catalyst system preferably in an amount of 0.01 to 25 parts by weight of rhodium per 100 parts by weight of support, preferably 0.02 to 8 parts by weight of rhodium per 100 parts by weight of support, this corresponds to a further preferred embodiment of the invention.

When the weight ratio of rhodium to ruthenium in the catalyst system is 0.03 to 15 parts by weight of rhodium per part by weight of ruthenium this in turn is a preferred embodiment of the invention.

The catalysts, in particular rhodium and/or ruthenium, may be added to the support for example either by wet impregnation or for example by precipitation in the presence of a base in water. Preferred bases would be LiOH, Li₂CO₃ or Na₂CO₃. The catalyst system preferably composed of rhodium, ruthenium and the support is advantageously dried and in particular heated to a temperature of <400° C.

The support for the catalyst, in particular rhodium and/or ruthenium, is preferably a lithium aluminate support, in particular spinel LiAl₅O₈. This is a known composition and known as a support for catalyst systems. This corresponds to a further preferred embodiment of the invention. Further possible supports are for example aluminium oxide and other metal oxide supports.

The support may preferably be produced by a solution process in which an aqueous lithium salt is mixed as a solution with aluminium oxide, silicon dioxide, zirconium oxide or titanium dioxide followed by drying and calcining, typically in air. The calcining is preferably carried out at temperatures in the range from 500° C. to 1500° C., in particular from about 700° C. to 1000° C., to ensure that the composition LiAl₅O₈ is obtained.

Typical calcining preferably requires at least 5 hours, in particular 10 to 25 hours. In the formulation of the lithium aluminium support the content of lithium salt is preferably controlled such that an atom ratio of the lithium/aluminium ratio of 0.2 to 1.5 to 5 may be provided.

When the weight ratio of ruthenium to lithium aluminate is 2 to 8 parts by weight of ruthenium per 100 part by weight of lithium aluminate this is a preferred embodiment of the invention.

As in conventional processes the hydrogenation according to the invention may preferably be performed under liquid-phase conditions. Liquid-phase conditions are typically brought about by performing the hydrogenation in the presence of a solvent. Although it is also possible to perform the reaction in the absence of a solvent, processing is normally much simpler when a solvent is used. Preferred solvents suitable for performing the hydrogenation according to the invention comprise saturated aliphatic and/or alicyclic hydrocarbons such as cyclohexane, hexane and/or cyclooctane; low molecular weight alcohols such as methanol, ethanol and/or isopropanol; and/or aliphatic and/or alicyclic hydrocarbon ethers, such as n-propyl ether, isopropyl ether, n-butyl ether, amyl ether, tetrahydrofuran, dioxane and/or dicyclohexyl ether. Tetrahydrofuran is the most preferred solvent.

It is most preferred in the context of the present invention when the catalyst is ruthenium and the support is aluminum oxide and when the hydrogenation is performed in the presence of solvent. This corresponds to a very particularly preferred embodiment of the invention. If the hydrogenation employs solvents, one or more solvents may be employed.

If a solvent is used it may preferably be used in amounts from 100 parts by weight based on 100 parts by weight of the aromatic amine introduced into the reaction and the solvent is preferably employed in amounts from about 200 to about 600 parts by weight based on 100 parts by weight of the aromatic amine introduced into the reaction.

The reaction temperature range in the process according to the invention is preferably between 130° C. and 210° C., in particular between 170° C. and 200° C.

It corresponds to a preferred embodiment of the invention when in the process according to the invention the hydrogenation pressure is 1.48 MPa to 27.68 MPa.

The reaction time in the process according to the invention varies according to the amount of impurities in the starting material to be hydrogenated but is preferably in the range from one hour to several days

The catalyst contents in the process according to the invention may preferably be in the range from 0.5% to 5% by weight of the starting material to be hydrogenated, i.e. of the aromatic amines to be hydrogenated.

According to the invention the input product comprising aromatic amines results from a polyurethane decomposition process and contains impurities from this decomposition process. The aromatic amines comprise in particular methylenedianiline and/or oligomers thereof and/or tolylenediamine. Tolylenediamine comprises in particular 2,4- or 2,6-tolylenediamine or any desired mixtures of these isomers Methylendianiline comprises in particular 4,4′-diaminodiphenylmethane, 2,2′-diaminodiphenylmethane or 2,4′-diaminodiphenylmethane or any desired mixtures of these isomers.

The polyurethane decomposition process may be any desired process which makes use of the known chemical processes such as hydrolysis, for example described in U.S. Pat. No. 5,208,379, glycolysis, acidolysis, aminolysis, hydrogenolysis, solvolysis or similar processes in customary fashion.

However it has been found in the context of the present invention in a particularly preferred embodiment of the invention that, surprisingly, the process according to the invention for catalytic hydrogenation has then proven very particularly advantageous when the input product comprising aromatic amines results from a PU decomposition process, which is based on a hydrolysis, in particular comprises the following steps of:
a) depolymerizing a polyurethane by hydrolysis in the presence of a base and a catalyst selected from the group consisting of quaternary ammonium salts containing an ammonium cation comprising 6 to 30 carbon atoms and organic sulfonates containing at least 7 carbon atoms at temperatures preferably below 200° C. to produce aromatic amines,
b) separating the organic phase comprising the aromatic amines from the aqueous phase,
c) optionally, preferably mandatorily, separating the aromatic amine from the organic phase, preferably by distillation or by extraction processes,
d) optionally, preferably mandatorily, reusing the separated aqueous phase in the depolymerization of a polyurethane by hydrolysis according to step a).

Since the abovementioned, particularly preferred, PU decomposition process which corresponds to a preferred embodiment of the invention is a hydrolysis the decomposition of the polyurethane affords a biphasic mixture comprising an aqueous phase and an organic phase. The organic phase includes the aromatic amines to be hydrogenated later and also impurities. The catalyst may be a quaternary ammonium salt containing an ammonium cation comprising 6 to 30 carbon atoms or an organic sulfonate containing at least 7 carbon atoms.

The corresponding, preferred hydrolysis processes of PU materials are for example described in the as yet unpublished European patent applications under filing numbers 20192354.7 (WO2022/042909A1) or 20192364.6 (WO2022/042910A1).

Workup may be effected by phase separation with customary methods. If no phase separation is performed the organic phase may also be separated by extraction.

It is a particular advantage of the process that the separated aqueous phase may be re-employed in the depolymerization of a polyurethane by hydrolysis according to step a), in particular without further workup.

After the separation of the organic phase comprising the aromatic amines, said amines may be obtained from the organic phase, for example distilled off.

A particularly preferred variant of the depolymerization, referred to here as preferred variant 1, is described below.

It is in particular preferable when the depolymerization of the polyurethane in step a) is effected using a base having a pK_{b} at 25° C. of 1 to 10, preferably 1 to 8, more preferably 1 to 7, in particular 1.5 to 6, and a catalyst selected from the group consisting of (i) quaternary ammonium salts containing an ammonium cation comprising 6 to 30 carbon atoms and (ii) organic sulfonates containing at least 7 carbon atoms. This corresponds to a preferred embodiment of the invention.

Preferred bases comprise an alkali metal cation and/or an ammonium cation. Preferred bases are alkali metal phosphates, alkali metal hydrogenphosphates, alkali metal carbonates, alkali metal silicates, alkali metal hydrogencarbonates, alkali metal acetates, alkali metal sulfites, ammonium hydroxides or mixtures of the above. Preferred alkali metals are Na, K or Li or mixtures of the above, in particular Na or K or mixtures thereof; a preferred ammonium cation is NH₄⁺.

Particularly preferred bases are K₂CO₃, Na₂SiO₃, NH₄OH, K₃PO₄ or KOAc

The base is preferably used as a saturated alkaline solution in water, wherein the weight ratio of saturated alkaline solution to PU is in the range from by preference 0.5 to 25, preferably 0.5 to 15, more preferably 1 to 10, in particular 2 to 7.

Preferred quaternary ammonium salts have the general structure: R₁R₂R₃R₄NX

where
R₁, R₂, R₃ and R₄ are identical or different hydrocarbon groups selected from alkyl, aryl and/or arylalkyl, wherein R₁ to R₄ are preferably selected such that the carbon atoms in the quaternary ammonium cation sum to 6 to 14, preferably 7 to 14, in particular 8 to 13.
X is selected from halide, preferably chloride and/or bromide, hydrogensulfate, alkyl sulfate, preferably methylsulfate or ethylsulfate, carbonate, hydrogencarbonate or carboxylate, preferably acetate or hydroxide.

Very particularly preferred quaternary ammonium salts are tributylmethylammonium chloride, tetrabutylammonium hydrogensulfate, benzyltrimethylammonium chloride, tributylmethylammonium chloride and/or trioctylmethylammonium methylsulfate. These are the most preferred catalysts.

The organic sulfonate containing at least 7 carbon atoms that is likewise employable as catalyst preferably comprises alkylaryl sulfonates, alpha-olefin sulfonates, petroleum sulfonates and/or naphthalene sulfonates.

Preferred temperatures for the depolymerization are 80° C. to 200° C., preferably 90° C. to 180° C., more preferably 95° C. to 170° C. and in particular 100° C. to 160° C.

Preferred reaction times for the depolymerization are 1 minute to 14 h, preferably 10 minutes to 12 h, preferably 20 minutes to 11 h and in particular 30 minutes to 10 h.

Preference is given to using for the depolymerization at least 0.5% by weight of catalyst based on the weight of the polyurethane, preferably 0.5% to 15% by weight, further preferably 1% to 10% by weight, even further preferably 1% to 8% by weight, further preferably still 1% to 7% by weight and in particular 2% to 6% by weight.

A preferred weight ratio of base to polyurethane is within the range from 0.01 to 50, preferably 0.1 to 25, in particular 0.5 to 20.

This related to the preferred variant 1 of the depolymerization.

A further particularly preferred variant of the depolymerization, referred to here as preferred variant 2, is described below

In a further preferred embodiment of the invention the depolymerization of the polyurethane in step a) is carried out using a base having a pK_{b} at 25° C. of <1, by preference 0.5 to −2, preferably 0.25 to −1.5, in particular 0 to −1, of a catalyst from the group of quaternary ammonium salts containing an ammonium cation having 6 to 14 carbon atoms when the ammonium cation does not comprise a benzyl radical or else containing an ammonium cation having 6 to 12 carbon atoms when the ammonium cation does comprise a benzyl radical.

Preferred bases are alkali metal hydroxides, alkali metal oxides, alkaline earth metal hydroxides, alkali metal oxides or mixtures thereof. Preferred alkali metals are Na, K or Li or mixtures of the above, in particular Na or K or mixtures thereof; preferred alkaline earth metals are Be, Mg. Ca, Sr or Ba or mixtures thereof, preferably Mg or Ca or mixtures thereof. A very particularly preferred base is NaOH.

Preferred quaternary ammonium salts have the general structure: R₁R₂R₃R₄NX

where
R₁, R₂, R₃ and R₄ are identical or different hydrocarbon groups selected from alkyl, aryl and arylalkyl.
X is selected from halide, preferably chloride and/or bromide, hydrogensulfate, alkyl sulfate, preferably methylsulfate or ethylsulfate, carbonate, hydrogencarbonate, carboxylate, preferably acetate or hydroxide.

Particularly preferred quaternary ammonium salts are here benzyltrimethylammonium chloride or tributylmethylammonium chloride.

Preferred temperatures for the depolymerization are 80° C. to 200° C., preferably 90° C. to 180° C., more preferably 95° C. to 170° C. and in particular 100° C. to 160° C.

Preferred reaction times for the depolymerization are 1 minute to 14 h, by preference 10 minutes to 12 h, preferably 20 minutes to 11 h and in particular 30 minutes to 10 h.

The depolymerization is preferably carried out using at least 0.5% by weight of catalyst based on the weight of the polyurethane, preferably 0.5% to 15% by weight, more preferably 1% to 10% by weight, yet more preferably 1% to 8% by weight, yet still more preferably 1% to 7% by weight and in particular 2% to 6% by weight.

A preferred weight ratio of base to polyurethane is in the range from 0.01 to 25, by preference 0.1 to 15, preferably 0.2 to 10, in particular 0.5 to 5.

Preference is given to using an alkaline solution comprising base and water, wherein the base concentration is preferably greater than 5% by weight, preferably 5% to 70% by weight, preferably 5% to 60% by weight, further preferably 10% to 50% by weight, even further preferably 15% to 40% by weight, in particular 20% to 40% by weight, based on the weight of the alkaline solution.

This related to preferred variant 2 of the depolymerization.

The PU to be recovered in the PU decomposition process may be any PU product, in particular comprising a polyurethane foam, preferably rigid PU foam, flexible PU foam, viscoelastic PU foam, HR PU foam, hypersoft PU foam, semirigid PU foam, thermoformable PU foam and/or integral PU foam.

### EXAMPLES

### Example 1 According to the Invention

A 1 L hydrogenation autoclave with a sparging stirrer was filled with a mixture of 121.7 g of recycled tolylenediamine* and 278.6 g of THF (INEOS, >99.9% purity). The reactor was inerted with nitrogen by three-fold pressure swing, whereafter 3.48 g of a ruthenium catalyst (15% Ru on aluminum oxide) were added to the stirred solution (1000 rpm) through an airlock. After renewed inerting by pressure swing the reactor was pressurized with 80 bar of hydrogen (Evonik Operations GmbH, >99.95% purity).

The reaction was commenced by increasing the temperature of the reactor to 185° C. over a period of 90 minutes. Over this time the reactor pressure increased to about 100 bar. This pressure was subsequently maintained through hydrogen control.

Samples were taken from the reaction through a sample tube after 60, 180, 300, 420 and 1380 min, without interrupting the reaction. The samples were analyzed by gas chromatography. The conversions and GC yields of methylcyclohexyldiamine (MCDA) achieved are shown in table 1.

### Recycled Tolylenediamine*:

The recycled tolylenediamine resulted from a PU decomposition process, based on a hydrolysis, performed according to claims 12 and 13 (after WO2022042909A1). The tolylenediamine produced according to claims 12 and 13 had a purity of 96.9% (GC %) (main secondary component 0.82% polyol).

### Example 2 According to the Invention

A 1 L hydrogenation autoclave with a sparging stirrer was filled with a mixture of 125.0 g of recycled tolylenediamine* and 280.9 g of THF (INEOS, >99.9% purity). The reactor was inerted with nitrogen by three-fold pressure swing, whereafter 3.50 g of a ruthenium catalyst (15% Ru on aluminum oxide) were added to the stirred solution (1000 rpm) through an airlock. After renewed inerting by pressure swing the reactor was pressurized with 80 bar of hydrogen (Evonik Operations GmbH, >99.95% purity)

The reaction was commenced by increasing the temperature of the reactor to 185° C. over a period of 90 minutes. Over this time the reactor pressure increased to about 100 bar. This pressure was subsequently maintained through hydrogen control.

Samples were taken from the reaction through a sample tube after 60, 180 and 1380 min, without interrupting the reaction. The samples were analyzed by gas chromatography. The conversions and GC yields of methylcyclohexyldiamine (MCDA) achieved are shown in table 1.

### Recycled Tolylenediamine*:

The recycled tolylenediamine resulted from a PU decomposition process, based on a hydrolysis, performed according to claims 12 and 13 (after WO2022042909A1). The tolylenediamine produced according to claims 12 and 13 had a purity of 91.29% (main secondary components: 0.79% polyol, 4.0% tributylamine).

### Comparative Example 1

A 1 L hydrogenation autoclave with a sparging stirrer was filled with a mixture of 96.5 g of 2,4-diaminotoluene (Sigma-Aldrich, 98% purity), 24.3 g of 2,6-diaminotoluene (Sigma-Aldrich, 97% purity) and 281.2 g of THF (INEOS, >99.9% purity). The reactor was inerted with nitrogen by three-fold pressure swing, whereafter 3.51 g of a ruthenium catalyst (15% Ru on aluminum oxide) were added to the stirred solution (1000 rpm) through an airlock. After renewed inerting by pressure swing the reactor was pressurized with 80 bar of hydrogen (Evonik Operations GmbH, >99.95% purity).

The reaction was commenced by increasing the temperature of the reactor to 185° C. over a period of 90 minutes. Over this time the reactor pressure increased to about 100 bar. This pressure was subsequently maintained through hydrogen control.

Samples were taken from the reaction through a sample tube after 60, 180, 360 and 1380 min, without interrupting the reaction. The samples were analyzed by gas chromatography. The conversions and GC yields of methylcyclohexyldiamine achieved are shown in table 1.

TABLE 1
Conversion of TDA (tolylenediamine) and yield
of MCDA of inventive and comparative example

| | Inventive example 1 | | Inventive example 2 | | Comparative example | |
|---|---|---|---|---|---|---|
| | Conver- | Yield of | Conver- | Yield of | Conver- | Yield of |
| Time | sion | MCDA | sion | MCDA | sion | MCDA |
| [min] | [GC %] | [GC %] | [GC %] | [GC %] | [GC %] | [GC %] |
| 0 | 3.12 | 0.09 | 0.15 | 0.11 | 0.19 | 0.04 |
| 60 | 15.16 | 11.66 | 7.14 | 6.47 | 13.10 | 11.53 |
| 180 | 65.44 | 56.99 | 53.40 | 45.58 | 68.36 | 60.74 |
| 300 | 84.68 | 74.12 | nd | | nd | nd |
| 360 | nd | nd | nd | | 93.89 | 83.81 |
| 420 | 94.11 | 82.69 | nd | | nd | nd |
| 1380 | 99.99 | 86.90 | 100 | 83.92 | 99.99 | 87.38 |

nd = not determined

1. A process for catalytic hydrogenation of aromatic amines, to afford their ring-hydrogenated equivalents, the process comprising:
contacting an input product, which comprises the aromatic amines and up to 25% by weight of impurities, with hydrogen in the presence of a catalyst applied to a support, wherein the input product comprising aromatic amines results from a polyurethane decomposition process and contains impurities from this decomposition process.

2. The process according to claim 1, wherein the catalyst comprises rhodium and/or ruthenium.

3. The process according to claim 2, wherein rhodium and ruthenium are employed together and a weight ratio of rhodium to ruthenium is 1 to 20 parts of rhodium per part of ruthenium.

4. The process according to claim 2, wherein the support is a lithium aluminate support.

5. The process according to claim 4, wherein a weight ratio of ruthenium to lithium aluminate is 2 to 8 parts by weight of ruthenium per 100 parts by weight of lithium aluminate.

6. The process according to claim 1, wherein a catalyst system is a physical mixture of rhodium on lithium aluminate and ruthenium on lithium aluminate.

7. The process according to claim 3, wherein a weight ratio of rhodium to ruthenium in the catalyst system is 6 to 15 parts by weight of rhodium per part by weight of ruthenium.

8. The process according to claim 1, wherein ruthenium supported on aluminum oxide (Al₂O₃) is employed as the catalyst.

9. The process according to claim 8, wherein a weight ratio of ruthenium to aluminum oxide is 4 to 25 parts by weight of ruthenium per 100 parts by weight of aluminum oxide.

10. The process according to claim 1, wherein the hydrogenation is performed in the presence of a solvent.

11. The process according to claim 1, wherein a hydrogenation pressure is 1.48 MPa to 27.68 MPa.

12. The process according to claim 1, wherein the polyurethane decomposition process comprises:
a) depolymerizing a polyurethane by hydrolysis in the presence of a base and at least one catalyst selected from the group consisting of quaternary ammonium salts containing an ammonium cation comprising 6 to 30 carbon atoms and organic sulfonates containing at least 7 carbon atoms to produce aromatic amines,
b) separating an organic phase comprising the aromatic amines from an aqueous phase,
c) optionally separating the aromatic amines from the organic phase, and
d) optionally reusing the separated aqueous phase in the depolymerization of a polyurethane by hydrolysis according to a).

13. The process according to claim 12, wherein the depolymerization of the polyurethane in a) is effected using a base having a pK_{b} at 25° C. of 1 to 10 and at least one catalyst selected from the group consisting of quaternary ammonium salts containing an ammonium cation comprising 6 to 30 carbon atoms and organic sulfonates containing at least 7 carbon atoms.

14. The process according to claim 12, wherein the depolymerization of the polyurethane in a) is carried out using a base having a pK_{b} at 25° C. of <1 and at least one catalyst selected from the group consisting of quaternary ammonium salts containing an ammonium cation having 6 to 14 carbon atoms when the ammonium cation does not comprise a benzyl radical and quaternary ammonium salts containing an ammonium cation having 6 to 12 carbon atoms when the ammonium cation does comprise a benzyl radical.

15. The process according to claim 1, wherein the polyurethane to be decomposed in the PU decomposition process comprises a polyurethane foam.

16. The process according to claim 1, wherein the aromatic amines comprise methylenedianiline and/or tolylenediamine.

17. The process according to claim 1, wherein the impurities are at least one selected from the group consisting of alcohols, glycols, polyols, organic acids, tertiary amines, quaternary amines, aldehydes, and water.

18. The process according to claim 1, wherein the catalyst is ruthenium.

19. The process according to claim 10, wherein the solvent is used in an amount from 100 parts by weight based on 100 parts by weight of the aromatic amine introduced into the reaction.

20. The process according to claim 10, wherein the solvent is employed in amounts from about 200 to about 600 parts by weight based on 100 parts by weight of the aromatic amine introduced into the reaction.
